(19) United States
(12) Patent Application Publication
(10) Pub. No.: US 2003/0057826 A1
(43) Pub. Date: Mar. 27, 2003

(54) Nitrogen-containing heterocyclic compound

(76) Inventors: Masaru Matsuoka, Tenri-shi (JP); Hiroshi Matsuoka, Tenri-shi (JP); Kazuko Shirai, Osaka (JP); Toru Kitaguchi, Yokohama-shi (JP); Shigeki Kambara, Himeji-shi (JP)

Correspondence Address: BIRCH STEWART KOLASCH & BIRCH, PO BOX 747, FALLS CHURCH, VA, 22040-0747, US

(21) Appl. No.: 10/143,760

(22) Filed: May 14, 2002

(30) Foreign Application Priority Data
May 14, 2001 (JP) 143744/2001

(51) Int. Cl. H01J 1/62; H01J 63/04; C07D 243/08; C 07D 4 3/14

(52) U.S. Cl. 313/504; 540/575

(57) ABSTRACT

A nitrogen-containing heterocyclic compound represented by the following formula (I):

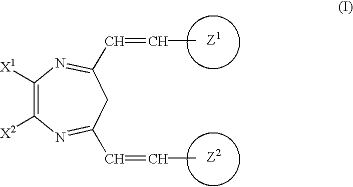

wherein X¹ and X² each represent an electron attractive group such as a cyano group; and the rings Z¹ and Z² each represent a hydrocarbon ring or a heterocycle (e.g., benzene ring having an electron donative group such as hydroxyl group, an alkoxy group, amino group and an N-substituted amino group, or a condensed ring being a benzene ring condensed with a heterocycle having a nitrogen atom as a hetero atom) is provided. An organic EL device is obtained by allowing an organic layer containing the compound described-above to be interposed between a pair of electrodes. The present invention provides a nitrogen-containing heterocyclic compound useful as a functional material and a process for producing the same, and an organic EL device using the same.

### FIELD OF THE INVENTION

The present invention relates to novel nitrogen-containing heterocyclic compounds useful as functional materials (particularly, materials for use in organic electroluminescence devices), a process for producing the same, and an organic electroluminescence device using the same.

### BACKGROUND OF THE INVENTION

Conventionally, a variety of pigments or dyes (e.g., azo-type, anthraquinone-type, and phthalocyanine-type pigments) have been in use for dying or coloring fiber and others. These pigments or dyes, taken advantage of their characteristic of having a π-electron conjugate bond within a pigment molecule, have been utilized as, so called, functional pigments in addition to the application of dying or coloring. Since these functional pigments have such functions as light-absorptivity (e.g., color, pleochroism), luminous radiation (e.g., fluorescent light), photoconductivity, and reversible changes by heat or light (e.g., thermochromism, photochromism), these have been used as functional materials in a variety of fields. For example, functional pigments that emit fluorescent light upon irradiation of light have been used as fluorescent materials such as fluorescent dyes, fluorescent pigments, fluorescent flaw detecting agents, and fluorescent white dyes. Moreover, pigments that show photochromism have been applied for use in photochromic photos, photochromic materials (light-adjusting materials) for sunglasses, and others. Since photochromic compounds develop colors or fade as their molecular structure changes by the action of light, these compounds have been utilized also as rewritable optical recording materials. These recording materials have high image resolution and thus do not need to be developed in their coloring reaction.

Among these functional pigments mentioned above, in particular, those that emit light by the action (application) of electric fields are useful as emission center compounds for use in organic electroluminescence devices (hereinafter, occasionally abbreviated simply as organic EL device).

Conventionally, organic electroluminescence devices are composed of a compound or compounds having an electron-transporting function, a hole-transporting function, and an emission center function. There have been reported single-layered ones in which a single layer is provided with all the functions mentioned above, and multi-layered ones in which layers have different functions. Its principle of light emission is considered to be based on the phenomenon that electrons and holes injected from a pair of electrodes recombine within a light-emitting layer to form excitons, exciting an emission center compound forming a light-emitting layer.

Colors that organic EL devices emit can be selected by suitably choosing an emission center compound constituting the light-emitting layer. For example, Japanese Patent Application Laid-Open No. 73443/1996 (JP-A-8-73443) discloses the dimer of pyrazine in which a pyrazine group having a phenyl group is bound to a divalent aromatic group, and an organic EL device containing this pyrazine derivative in an organic layer thereof. However, since this pyrazine dimer emits blue light of which the wavelength is relatively short, the electroluminescence device is limited in its emission wavelength and thus has greatly limited applications.

Incidentally, in The 3rd International Symposium on Photochromism (the date: Nov. 14 through 18, 1999, the sponsor: The 3rd International Symposium on Photochromism organizing committee, the cosponsor: The Chemical Society of Japan), a certain fluorescent azepine dye and a spectroscopic property thereof have been reported.

### SUMMARY OF THE INVENTION

Thus, it is an object of the present invention to provide a compound capable of emitting light upon irradiation of light or by the action of electric fields and useful as a functional material such as an organic EL device-use material, a process for producing the same, and an organic EL device using the same.

Another object of the present invention is to provide a compound of which the emission wavelength is controllable over a wide range and capable of emitting light of longer wavelength (e.g., yellow to red light), and an organic EL device using the same.

The inventors of the present invention made intensive studies to achieve the above objects and finally found that a specific heterocyclic compound containing a nitrogen atom as a hetero atom emits light upon irradiation of light or by the action of electric fields and therefore is useful as such a functional material as those for organic electroluminescence devices. The present invention was accomplished based on the above findings.

That is, the nitrogen-containing heterocyclic compound of the present invention is represented by the following formula (I):

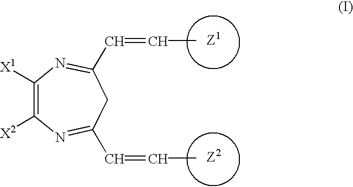

wherein X¹ and X² are the same or different, each representing an electron attractive group; and the rings Z¹ and Z² are the same or different, each representing a hydrocarbon ring which may have a substituent or a heterocycle which may have a substituent.

The electron attractive group may be selected from a cyano group, a carbonyl group, an acyl group, and a carboxyl group. At least X¹ or X² may be a cyano group. Each of the rings Z¹ and Z² may be bonded to the adjacent C═C bond to form a conjugated system. The rings Z¹ and Z² may be the same or different, each representing an aromatic ring (e.g., benzene ring) having an electron donative group, or a condensed ring which has an electron donative group and is condensed an aromatic hydrocarbon ring (e.g., benzene ring) with a heterocycle having a nitrogen atom as a hetero atom. Each of the electron donative groups may be selected from a hydroxyl group, an alkoxy group, an amino group and an N-substituted amino group. The heterocycle constituting the condensed ring may be 5- or 6-membered heterocycle. The substituent of the rings Z¹ and Z² may be at least one member selected from the group consisting of an alkyl group, a cycloalkyl group, an aryl group, an aralkyl group, a halogen atom, a hydroxyl group, an alkoxy group, a mercapto group, an alkylthio group, a hydroxyalkyl group, a carbonyl group, a carboxyl group, an alkoxycarbonyl group, an acyl group, an acyloxy group, a cyano group, an amino group, an N-substituted amino group, a nitro group, and a sulfonyl group. The compound is capable of emitting light by applying a light (e.g., a light having a wavelength of 360 to 860 nm) or an electric field, and may emit fluorescent light.

The present invention further includes a process for producing a compound represented by the above-mentioned formula (I). The present invention further includes an organic electroluminescence device having, between a pair of electrodes, an organic layer (e.g., light-emitting layer) comprising a compound represented by the formula (I) shown above. The organic layer of this organic electroluminescence device may have (1) a single-layered structure composed of a light-emitting layer having at least one function selected from an electron-transporting function and a hole-transporting function, or (2) a multilayered structure (lamination) composed of a layer having at least one function selected from an electron-transporting function and a hole-transporting function, and a light-emitting layer. Moreover, the organic layer may comprise a compound represented by the above-shown formula (I) and an organic polymer having at least one function selected from an electron-transporting function and a hole-transporting function.

### BRIEF DESCRIPTION OF THE DRAWINGS

FIG. 1 shows the fluorescence spectrum of the organic electroluminescence device obtained in Example 5.

FIG. 2 is a graph showing the emission luminance of the organic electroluminescence devices obtained in Example 5 versus voltage applied thereto.

### DETAILED DESCRIPTION OF THE INVENTION

The compound of the present invention (hereinafter, sometimes referred to as a nitrogen-containing heterocyclic compound) is represented by the following formula (I):

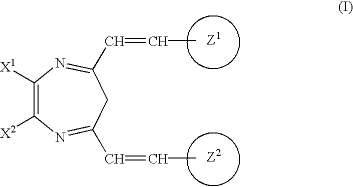

wherein X¹ and X² are the same or different, each representing an electron attractive group; and rings Z¹ and Z² are the same or different, each representing a hydrocarbon ring which may have a substituent or a heterocycle which may have a substituent.

Exemplified as the electron attractive groups represented by X¹ and X² are a cyano group, a carbonyl group, an acyl group and a carboxyl group, and others. As the electron attractive groups, a cyano group is preferred. Usually, at least one of X¹ and X² is a cyano group, and it is preferred that both of which are cyano groups. A nitrogen-containing heterocycle having such X¹ and X² (e.g., dicyanoazepine ring) seems to function as a coloring system upon intramolecular charge transfer.

Although the hydrocarbon ring represented by each of the rings Z¹ and Z² may be a non-aromatic hydrocarbon ring (e.g., C₃₋₂₀ cycloalkanes such as cyclohexane and cyclooctane, C₃₋₂₀ cycloalkenes such as cyclohexene), the hydrocarbon ring is usually an aromatic hydrocarbon ring. The aromatic hydrocarbon ring has at least a benzene ring in most case, examples of which are benzene ring and condensed polycyclic aromatic hydrocarbon rings (e.g., a condensed polycyclic aromatic hydrocarbon ring having 6 to 20 carbon atoms, preferably 6 to 14 carbon atoms, and more preferably 6 to 10 carbon atoms, such as naphthalene ring, anthracene ring, phenanthrene ring, phenalene ring). Preferred as the hydrocarbon ring is benzene, naphthalene, and phenalene rings.

Included among the examples of the heterocycle represented by each of the rings Z¹ and Z² are heterocycles containing at least one hetero atom selected from nitrogen, oxygen, and sulfur atoms, and these may be condensed (ortho-condensed, ortho and peri-condensed) heterocycles of a plurality of heterocycles condensed together or of a heterocycle condensed with a hydrocarbon ring (non-aromatic hydrocarbon rings, or aromatic hydrocarbon rings), not limited to monocyclic heterocycles. Although it does not matter if the heterocycle is non-aromatic, it is usually aromatic.

Examples of the heterocycle having a nitrogen atom as a hetero atom are: a 5- or 6-membered monocyclic heterocycle such as pyrrole, imidazole, pyridine, and pyrazine rings; a condensed heterocycle being a hydrocarbon ring (e.g., benzene ring) condensed with a 5- or 6-membered heterocycle such as indole, indoline, quinoline, isoquinoline, quinoxaline, quinazoline, carbazole, phenanthridine, acridine, and phenazine rings. In the condensed heterocycle, a nitrogen atom as a hetero atom may be situated in a position in which the hydrocarbon ring is condensed with the 5- or 6-membered heterocycle. As the heterocycle having an oxygen atom as a hetero atom, there are exemplified a5- or 6-membered monocyclic heterocycle such as furan ring; and a condensed heterocycle being a hydrocarbon ring (e.g., benzene ring) condensed with a 5- or 6-membered heterocycle such as isobenzofuran ring and chromene ring. Included among the examples of the heterocycle having a sulfur atom as a hetero atom are a 5- or 6-membered monocyclic heterocycle such as thiophene ring; and a condensed heterocycle being a hydrocarbon ring (e.g., benzene ring) condensed with a 5- or 6-membered heterocycle such as thianthrene ring. Exemplified as heterocycles having hetero atoms of different kinds are: a 5- or 6-membered monocyclic heterocycle such as morpholine, isothiazole, and isoxazole rings; and a condensed heterocycle being a hydrocarbon ring (e.g., benzene ring) condensed with a 5- or 6-membered heterocycle such as phenoxathiin ring.

Preferred heterocycles include: an aromatic heterocycle such 5- or 6-membered heterocycles having a nitrogen atom as a hetero atom (e.g., pyrrole ring, pyridine ring); an aromatic heterocycle (e.g., carbazole ring) being an aromatic hydrocarbon ring (particularly, benzene ring or naphthalene ring) condensed with a 5- or 6-membered heterocycle having at least a nitrogen atom as a hetero atom; and an aromatic heterocycle being an aromatic hedrocarbon ring (particularly, benzene ring or naphthalene ring) condensed (ortho and peri-condensed) with a heterocycle having a nitrogen atom as a hetero atom (e.g., piperidinopiperazine ring) in a position in which the condensation occurs.

Each of the rings Z¹ and Z² is usually bonded to the adjacent C═C bond at the aromatic ring constituting the ring Z¹ or Z² to form a conjugated system. Moreover, in the case of a polycyclic ring, insofar as the aromatic ring is bonded to the adjacent C═C bond, it does not matter whether the other ring or rings are non-aromatic or aromatic ones, and part of the ring may be hydrogenated. As the hydrocarbon ring partially hydrogenated, there are mentioned hydrogenated naphthalene rings such as 1,2-dihydronaphthalene, 2,3-dihydrophenalene and 2,3,3a,4,5,6-hexahydrophenalene rings. Moreover, as the heterocycle partially hydrogenated, there are mentioned julolidine ring and 9-formyljulolidine ring.

The rings Z¹ and Z² may have a variety of substituents, examples of which are alkyl groups (e.g., C₁₋₆alkyl groups typified by methyl and ethyl groups); cycloalkyl groups (e.g., C₃₋₁₀cycloalkyl groups); aryl groups (e.g., C₆₋₁₈aryl groups typified by phenyl group); arylalkyl (or aralkyl) groups (e.g., C₆₋₁₂aryl-C₁₋₄alkyl groups typified by benzyl and diphenylmethyl groups); halogen atoms (fluorine, chlorine, bromine, and iodine atoms); hydroxyl group; alkoxy groups (e.g., C₁₋₆alkoxy groups such as methoxy, ethoxy, propoxy, isopropoxy, butoxy, isobutoxy, s-butoxy and t-butoxy groups); mercapto group; alkylthio groups corresponding to the above-mentioned alkoxy groups; hydroxyalkyl groups (e.g., hydroxyC₁₋₆alkyl groups typified by hydroxymethyl group); carbonyl group; carboxyl group; alkoxycarbonyl groups (e.g., C₁₋₄alkoxy-carbonyl groups such as ethoxycarbonyl group); acyl groups (e.g., C₁₋₆alkyl-carbonyl groups, C₆₋₁₂aryl-carbonyl groups); acyloxy groups (e.g., C₁₋₆acyloxy groups typified by acetyloxy group); cyano group; amino group; N-substituted amino groups (e.g., mono- or diC₁₋₆alkylamino groups typified by methylamino, dimethylamino, diethylamino, methylethylamino and dibutylamino groups, mono- or diC₆₋₁₈arylamino groups typified by phenylamino group, C₁₋₆acylamino groups typified by acetamide group); nitro group; and sulfonyl group.

Preferred substituents include C₁₋₄alkyl groups, C₆₋₁₂aryl groups, hydroxyl group, C₁₋₄alkoxy groups, C₁₋₄acyloxy groups, amino group, and N-substituted amino groups (e.g., mono- or diC₁₋₆alkylamino groups, mono- or diC₆₋₁₈arylamino groups and C₁₋₄acylamino groups). Particularly, as the substituent(s), an electron donative group [e.g., hydroxyl group, alkoxy groups (such as C₁₋₄alkoxy groups), amino group, N-substituted amino groups (such as mono- or diC₁₋₄alkylamino groups)] is preferable.

There is no particular restriction as to the position(s) of the substituent(s) on the ring Z¹ and Z² each. For example, on the benzene ring, the substituent(s) is/are in the o-, m-, or p-position, and usually in the o- and/or p-position. Moreover, the rings Z¹ and Z² each may have a plurality of substituents.

Each of the preferred rings Z¹ and Z² is an aromatic ring having an electron donative group, a condensed ring being an aromatic hydrocarbon ring (particularly, benzene ring) condensed with a heterocycle (a 5- or 6-memberd heterocycle) having a nitrogen atom as a hetero atom. The nitrogen atom as a hetero atom may have a C₁₋₆alkyl group as a substituent. Exemplified as such rings Z¹ and Z² are benzene ring having a substituent(s) (e.g., benzene rings substituted with at least one member selected from a hydroxyl group, a C₁₋₄alkoxy group, an amino group, and a mono- or diC₁₋₄alkyl-substituted amino group); an N-substituted heterocycle in which the nitrogen atom as a hetero atom may be substituted with a C₁₋₆alkyl group [e.g., carbazole ring which may be substituted with an N-C₁₋₄alkyl group]; and a condensed heterocycle being benzene ring ortho and peri-condensed with a condensed heterocycle having a nitrogen atom as a hetero atom in a position in which the condensation occurs (e.g., julolidine ring).

In the formula (I), examples of combinations of substituents are exemplified below.

X¹: cyano group

X²: cyano group

Z¹ and Z²: an aromatic hydrocarbon ring (particularly, benzene ring) or a condensed polycyclic hydrocarbon ring, each of which has no substituents or has a substituent [at least one member selected from a hydroxyl group, an alkoxy group (particularly, a C₁₋₄alkoxy group), an amino group, and an N-substituted amino group]; or a polycyclic condensed heterocycle being an aromatic hydrocarbon ring (particularly, benzene ring) condensed with a heterocycle containing a nitrogen atom as a hetero atom (particurarly, a 5- or 6-membered heterocycle).

Typical examples of the compound represented by the formula (I) (a diazepine ring-containing compound) are 2,3-dicyano-5,7-bis(phenyl)vinyl-1-yl-6H-1,4-diazepine; 2,3-dicyano-5,7-bis(hydroxyphenyl)vinyl-1-yl-6H-1,4-diazepine; 2,3-dicyano-5,7-bis (C₁₋₄alkoxyphenyl)vinyl-1-yl-6H-1,4-diazepine; 2,3-dicyano-5,7-bis(mono- or diC₁₋₄alkylaminophenyl)vinyl-1-yl-6H-1,4-diazepine; 2,3-dicyano-5,7-bis(carbazolyl)vinyl-1-yl-6H-1,4-diazepine; and 2,3-dicyano-5,7-bis(julolidinyl)vinyl-1-yl-6H-1,4-diazepine.

[Production Process]

The compound of the present invention can be prepared in accordance with the following reaction formula (1).

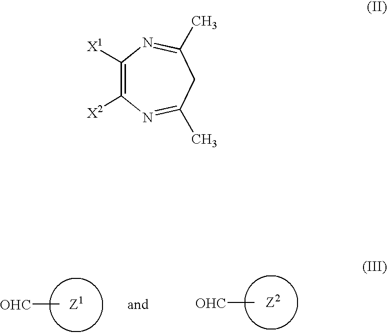

wherein X¹, X², the ring Z¹ and the ring Z² have the same meanings as defined above.

The compound of the formula (I) can be obtained by reacting the compound represented by the formula (II) (including its structural isomers) with the compound represented by the formula (III) (aldehydes). Probably due to the C═C double bond in the nitrogen-containing heterocycle of the compound of the formula (II) isomerized into an enamine, it is possible to react the compound of the formula (II) with an aldehyde (III) efficiently.

Typical examples of the compound represented by the formula (III) are aldehydes corresponding to the above-mentioned rings Z¹ and Z² [e.g., aldehydes in which each of the rings Z¹ and Z² is a benzene ring (e.g., benzaldehyde, halobenzaldehyde, aminobenzaldehyde, N-substituted aminobenzaldehyde, phenol-aldehyde, C₁₋₄alkoxybenzaldehyde); aldehydes in which each of the rings Z¹ and Z² is a condensed polycyclic hydrocarbon ring (e.g., naphthalenecarbaldehyde, phenalenecarbaldehyde); aldehydes in which each of the rings Z¹ and Z² is a 5- or 6-membered heterocycle containing a nitrogen atom as a hetero atom, or a condensed heterocycle of a heterocycle and a hydrocarbon ring (e.g., 9-alkyl-3-formylcarbazole)]. The amount of the compound represented by the formula (III) is, per 1 mol of the compound of the formula (II), about 1 to 3 mol, preferably about 1 to 1.5 mol.

The reaction described above may be carried out in the presence of a solvent inert to the reaction, such as an aliphatic hydrocarbon (e.g., hexane), an alicyclic hydrocarbon (e.g, cyclohexane), an aromatic hydrocarbon (e.g., benzene, toluene), a halogenated hydrocarbon (e.g., chloroform), an alcohol (e.g., methanol, ethanol, isopropyl alcohol, butanol), an ester (e.g., ethyl acetate, butyl acetate, isobutyl acetate), an ether (e.g., dioxane, diethyl ether, teterahydrofuran), an amide (formamide, acetamide, dimethylformamide (DMF), dimethylacetamide), a nitrile (e.g., acetonitrile, benzonitrile), a sulfoxide (e.g., dimethyl sulfoxide). If necessary, a catalyst (e.g., a basic catalyst such as pyridine and piperidine) may be used. The amount of the catalyst to be used can be selected within the range of, per 1 mol of the compound of the formula (II), about 0.001 to 1 mol.

When a solvent is used, the reaction temperature can be selected within the range of about 0° C. to reflux temperature and is for example about 50 to 120° C., preferably 60 to 100° C. The reaction can be effected under ordinary pressure, reduced pressure, or applied pressure. The reaction may be carried out in an atmosphere of an inert gas (e.g., nitrogen, argon, helium).

After completion of the reaction, the compound (I) formed in the above-described reaction can easily be separated and purified by such a conventional means as filtration, condensation, distillation, extraction, crystallization, recrystallization, column chromatography, or a combination means thereof.

The compound represented by the formula (II) may be prepared also in accordance with the following reaction formula (2).

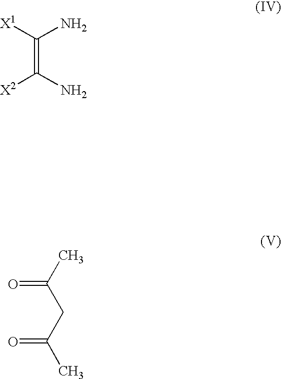

wherein X¹ and X² have the same meanings as defined above.

The compound of the formula (II) can be formed by reacting (dehydration-condensing) the compound represented by the formula (IV) with the compound represented by the formula (V).

Typical examples of the compound represented by the formula (IV) are diamines [e.g., 1,2-dicyano-1,2-diaminoethene (diaminomaleonitrile), 1-cyano-2-(dimethylamino)-1,2-diaminoethene, 1,2-dicyano-2-(benzylamino)-1-aminoethene)].

Typical examples of the compound represented by the formula (V) are alkanediketone compounds [e.g., acetylacetone (pentane-2,4-dione)].

The amount of the compound of the formula (V) to be used is usually about 1 to 3 mol, preferably about 1 to 2 mol (e.g., about 1 to 1.5 mol) relative to 1 mol of the compound of the formula (IV).

The condensation reaction described above may be effected in the presence or absence of a catalyst. Exemplified as the catalyst are conventional ones, such as acid catalysts (e.g., inorganic acids such as sulfuric acid, phosphoric acid, hydrochloric acid; organic acids such as acetic acid, sultonic acid, p-toluenesulfonic acid) and basic catalysts (e.g., hydroxides or oxides of alkaline or alkaline earth metals). The amount of the catalyst to be used can be selected within the range of about 0.001 to 1 mol relative to 1 mol of the compound of the formula (IV). A dehydrating agent (e.g., phosphorus pentoxide) may additionally be used.

The condensation reaction may be effected in a solvent inert to the reaction. As the solvent, those listed above are available (e.g., toluene). When a solvent is used, the reaction temperature can be selected within the range of about 0° C. to reflux temperature, and is for example about 50 to 120° C., preferably about 60° C. to 100° C. It is possible to effect the reaction under ordinary, reduced, or applied pressure. The reaction may be effected in an atmosphere of an inert gas (e.g., nitrogen, argon, helium). After the reaction, the compound formed by the condensation reaction described above can easily be separated and purified by any separating means of those mentioned above.

The compound of the present invention is characterized in that, due to its specific structure represented by the formula (I), it is capable of emitting light by being supplied with energy externally (irradiation of light, the action of an electric field). There is no particular restriction as to the irradiation of light so far as light is of a certain wavelength capable of exciting the nitrogen-containing heterocyclic compound of the formula (I). For example, ultraviolet rays (400 nm or less) and visible rays (e.g., 360 to 860 nm (particularly, 400 to 760 nm)), particularly visible rays, can be used. The emission wavelength varies over a wide range (e.g., about 400 to 700 nm (violet to red)), depending on, for example, the kind(s) of the substituent(s) and the position of substitution. The nitrogen-containing heterocyclic compound (I) of the present invention presents, though varies for different kinds of substituents or positions of substitution, violet to red color (particularly, yellow to red) and has a large molar absorption coefficient. This may be because the composition is a color (developing) system of the intramolecular charge transfer type in which the nitrogen-containing heterocycle within its molecule and the aromatic ring being the rings Z¹ and Z² act as an electron attractive group (acceptor) and an electron donative group, respectively.

Upon irradiation of light (particularly, visible rays), the compound of the formula (I) emits fluorescent light in a solution. The wavelength of fluorescence varies within the range mentioned above. Particularly, although the nitrogen-containing heterocycle of the compound of the present invention has a non-planar structure, generally, it seems to have a tendency to emit fluorescent light of relatively long wavelengths (about 500 to 700 nm, preferably about 530 to 700 nm: yellow to red), probably because the heterocycle acts as a strong electron-withdrawing group.

Under the action of an electric field (injection of a carrier), the compound of the present invention is capable of emitting light. The emission wavelength can be selected within the range mentioned above. Moreover, the compound of the present invention is capable of emitting light of relatively long wavelengths (about 500 to 700 nm, preferably 550 to 700 nm: yellow to red). Therefore, the compound of the present invention is useful as an emission center compound for an organic EL device.

Moreover, the compound of the formula (I) in the form of a solid (e.g., a thin film on which the composition is vapor-deposited) shows the same properties (emission of light upon irradiation of light or by the action of an electric field) as those shown in the case of the compound being in a solution or in the form of liquid. Therefore, the composition of the present invention can be used not only in a liquid state but also in a solid state (e.g., as a film, powder, particles), and its use is not restricted.

So that the compound of the present invention is capable of emitting light upon irradiation of light or by the action of an electric field, it can be utilized in various fields as a functional material. For example, the compound of the present invention is useful not only as a fluorescent material (e.g., a fluorescent pigment, a fluorescent flaw detecting agent, a fluorescent white dye, particularly as a fluorescent material such as a fluorescent dye) but also as a material for display devices (e.g., light emitting device material such as an electroluminescence material).

[Organic electroluminescence device]

The electroluminescence (EL) device of the present invention is composed of a pair of electrodes and an organic layer interposed therebetween. The organic layer comprises at least the compound represented by the aforementioned formula (I). Particularly, the layer containing the compound of the formula (I) forms a light-emitting region, constituting a light-emitting layer. The light-emitting layer may be formed of a film-formable compound of the formula (I) singly, or of a film-formable or non-film-formable compound and a binder having a film-forming property. As the binder, a resin having a film-forming property (a thermoplastic resin, a thermosetting resin) can be usually employed.

Examples of the thermoplastic resin are olefinic resins such as polyethylene, polypropylene, ethylene-propylene copolymer, and polybutene; styrenic resins such as polystyrene, rubber-modified polystyrene (e.g., HIPS), acrylonitrile-styrene copolymer, and acrylonitrile-butadiene-styrene copolymer; acrylic resins [e.g., homo- or copolymers of (meth)acrylic monomers (e.g., C₁₋₆alkyl (meth)acrylates such as methyl (meth)acrylate, ethyl (meth)acrylate, and butyl (meth)acrylate; hydroxyC₂₋₄alkyl (meth)acrylates such as hydroxyethyl (meth)acrylate and hydroxypropyl (meth)acrylate; glycidyl (meth)acrylate; (meth)acrylic acid; (meth)acrylonitrile); copolymers of the (meth)acrylic monomers mentioned above with copolymerizable monomers (e.g., aromatic vinyl monomers such as styrene) (e.g., methyl methacrylate-styrene copolymer)]; vinyl-series resins such as vinyl alcohol-series polymers typified by polyvinyl alcohol and ethylene-vinyl alcohol copolymer, polyvinyl chloride, vinyl chloride-vinyl acetate copolymer, polyvinylidene chloride, polyvinyl acetate, and ethylene-vinyl acetate copolymer; polyamide-series resins such as 6-nylon, 6,6-nylon, 6,10-nylon, and 6,12-nylon; polyester resins [e.g., alkylene arylate-series resins such as polyalkylene terephthalates (e.g., polyethylene terephthalate, polybutylene terephthalate) and polyalkylene naphthalate, or alkylene arylate copolyester resins]; fluorine-containing resins; polycarbonate; polyacetal; polyphenylene ether; polyphenylene sulfide; polyether sulfone; polyether ketone; thermoplastic polyimide; thermoplastic polyurethane; and norbornene-series polymer.

Exemplified as the thermosetting (thermosettable) resin are phenolic resins, amino resins (e.g., urea resins, melamine resins), thermosetting acrylic resins, unsaturated polyester resins, alkyd resins, diallyl phthalate resins, epoxy resins, and silicone resins.

These binders can be used either singly or in combination. The content of the compound of the formula (I) is, per 100 parts by weight of the binder, about 0.01 to 10 parts by weight, preferably about 0.05 to 5 parts by weight, and more preferably about 0.1 to 3 parts by weight.

If necessary, into the light-emitting layer may be incorporated other emission center compounds. As the other emission center compounds, a compound which has a function as an emission center compound for an organic electroluminescence device and is capable of absorbing laser beams, in particular, a compound which is excited by an electron and/or a hole (positive hole) and emits light, can be used. Examples of the emission center compounds are heterocyclic compounds containing at least one hetero atom selected from oxygen, nitrogen, and sulfur atoms [e.g., bis(C₁₋₆alkyl-benzoxazoyl)thiophene typified by 2,5-bis(5-tert-butyl-2-benzoxazoyl)-thiophene; nile red; coumarins typified by coumarin 6 and coumarin 7; 4-(dicyanoC₁₋₄alkylene)-2-C₁₋₄alkyl-6-(p-diC₁₋₄alkylaminostyryl)-4H-pyran typified by 4-(dicyanomethylene)-2-methyl-6-(p-dimethylaminostyryl)-4H-pyran; and quinacridone]; condensed polycyclic hydrocarbons such as rubrene and perylene; tetraC₆₋₁₂aryl-1,3-butadiene such as 1,1,4,4-tetraphenyl-1,3-butadiene (TPB); bis(2-(4-C₁₋₄alkylphenyl)C₂₋₄alkynyl)benzene such as 1,4-bis(2-(4-ethylphenyl)ethynyl)benzene; and bis(2,2′-diC₆₋₁₂-arylvinyl)biphenyl such as 4,4′-bis(2,2′-diphenylvinyl)biphenyl. Of these, nile red and coumarin 6 are preferable. These emission center compounds can be used either singly or in combination.

The content of the emission center compound is selected within a range not adversely affecting the efficiency of emission of the compound (I) and about 0.01 to 10 parts by weight, about 0.05 to 5 parts by weight, and more preferably about 0.1 to 3 parts by weight relative to 100 parts by weight of the binder. The proportion of the compound of the formula (I) to the other emission center compound(s) (weight ratio) is about 40/60 to 100/0, preferably about 50/50 to 95/5, and more preferably about 60/40 to 90/10.

If necessary, the light-emitting layer comprising the compound of the formula (I) may be given an electron-transporting function and/or a hole-transporting function. For the purpose of giving such function(s), (1) to the light-emitting layer may be added organic polymers or compounds having the functions described above; or (2) the light-emitting layer may be laminated with a layer or layers having the functions described above. In the case (1), it is possible to fabricate an organic electroluminescence device having a single-layered structure.

Exemplified as the organic polymer having at least one function selected from the electron-transporting and hole-transporting functions are vinyl-series polymers having at least one functional group selected from hole-transporting functional groups and electron-transporting functional groups in the main chain or a side chain, such as polyphenylenevinylenes [e.g., homo- or copolymers of C6-12arylenevinylenes which may have a substituent (e.g., C₁₋₁₀alkoxy group), such as polyphenylenevinylene, poly(2,5-dimethoxyphenylenevinylene, and polynaphthalenevinylene]; polyphenylenes (particularly, polyparaphenylenes) [e.g., homo- or copolymers of phenylenes which may have a substituent (e.g., C₁₋₁₀ alkoxy groups), such as polyparaphenylene and poly-2,5-dimethoxyparaphenylene]; polythiophenes [e.g., polyC₁₋₂₀alkylthiophenes such as poly(3-alkylthiophene); polyC₃₋₂₀cycloalkylthiophenes such as poly(3-cyclohexylthiophene); homo- or copolymers of C6-20arylthiophenes which may have a substituent (e.g., C₁₋₁₀alkyl groups) such as poly(3-(4-n-hexylphenyl) thiophene); polyfluorenes such as polyC₁₋₂₀alkylfluorenes; vinyl-series polymers having at least one functional group selected from a hole-transporting functional group and an electron-transporting functional group in the main or side chain, such as poly-N-vinylcarbazole (PVK), poly-4-N,N-diphenylaminostyrene, poly(N-(p-diphenylamino)phenylmethacrylamide), poly(N,N′-diphenyl-N,N′-bis(3-methylphenyl)-1,1′-biphenyl-4,4′-diaminomethacrylamide) (PTPDMA), and poly-4-(5-naphthyl-1,3,4-oxadiazole)styrene; polyC₁₋₄alkylphenylsilanes such as polymethylphenylsilane; polymers derived from an aromatic amine derivative in the side or main chain; and copolymers thereof.

These organic polymers can be used either singly or in combination. Particularly preferred are homo- or copolymers of which the main component (e.g., 50 to 100% by weight) is N-vinylcarbazole (e.g., copolymers of those with copolymerizable monomers such as (meth)acrylic monomers, styrenic monomers, and vinyl ester-series monomers) and aromatic amine derivatives.

PVK is amorphous and excellent in heat resistance (glass transition temperature Tg: 224° C.). Although there is no particular restriction on the degree of polymerization of the PVK, it is for example about 100 to 1,000, preferably about 200 to 800.

In the case where the light-emitting layer is comprised of the compound of the formula (I) and the organic polymer described above, the content of the compound of the formula (I) is, per 100 parts by weight of the organic polymer, about 0.01 to 10 parts by weight, preferably about 0.05 to 5 parts by weight, and more preferably about 0.1 to 3 parts by weight.

If necessary, to the light-emitting layer comprised of the compound of the formula (I) and the organic polymer may be added a compound having an electron- or hole-transporting function.

Examples of the compound having an electron-transporting function are oxadiazole derivatives [e.g., oxadiazole derivatives having a C₆₋₂₀aryl group which may have a substituent, such as 2-(4-biphenyl)-5-(4-tert-butylphenyl)-1,3,4-oxadiazole (PBD), 2,5-bis(1-naphtyl)-1,3,4-oxadiazole (BND), 1,3-bis[5-(4-tert-butylphenyl)-1,3,4,-oxadiazole)]benzene (BPOB), 1,3,5-tris[5-(4-tert-butylphenyl)-1,3,4-oxadiazole]benzene (TPOB), and 1,3,5-tris[5-(1-naphtyl)-1,3,4-oxadiazole]benzene (TNOB); diphenoquinones [e.g., diphenoquinones which may have a substituent (e.g., C1-10 alkyl groups), such as 3,5,3′,5′-tetrakis-tert-butyldiphenoquione]1,2,3,4,5-pentaphenyl-1-3-cyclopentadiene (PPCP); and quinolinic acid complexes such as tris(8-quinolinorato) aluminium (III) complex, bis(benzoquinolinorato)beryllium complex, and tris(10-hydroxybenzo[h]quinolilate)beryllium complex, with oxadiazole derivatives (e.g., PBD) partcicularly preferred.

As the compound having a hole-transporting function, there may be exemplified aromatic tertiary amines such as N,N′-diphenyl-N,N′-bis(3-methylphenyl)-1,1 ′-biphenyl-4,4′-diamine (TPD), N,N′-diphenyl-N,N′-bis(1-naphthyl)-1,1′-biphenyl-4,4′-diamine (NPD), 1,1-bis[(di-4-tolylamino)phenyl]cyclohexane, N,N,N′N′-tetra(3-methylphenyl)-1,3-diaminobenzene (PDA), 4,4′,4″-tris(3-methylphenylphenylamino) triphenylamine (m-MTDATA), 4,4′,4,″-tris(1-naphthylphenylamino) triphenylamine(1-TNATA), 4,4′,4″-tris(2-naphthylphenylamino) triphenylamine (2-TNATA), 4,4′,4″-tri(N-carbazolyl)triphenylamine (TCTA), 1,3,5-tris[4-(3-methylphenylphenylamino)phenyl]benzene (m-MTDAPB), and triphenylamine; and phthalocyanines.

The compounds having an electron-transporting function and those having a hole-transporting function may be used either singly or in combination.

The content of the compound having an electron and/or hole-transporting function is, relative to 100 parts by weight of the binder (and/or the organic polymer described above), about 10 to 200 parts by weight, preferably about 30 to 150 parts by weight, and more preferably about 50 to 130 parts by weight.

When the light-emitting layer is lacking in either of the electron-transporting function or the hole-transporting function, or attempting to improve each function, a layer or layers having the desired function may be applied onto the light-emitting layer by a conventional method (e.g., vapor deposition, solution coating). These layers may be composed of low molecular weight compounds or high molecular weight compounds.

The thickness of each layer constituting the organic layer is not particularly limited, and is for example about 1 nm to 1 μm, preferably about 5 to 800 nm, more preferably about 10 to 500 nm, and particularly about 15 to 300 nm.

As the anode of the organic EL device, for example, a transparent electrode (e.g., indium-tin-oxide (ITO) electrode) formed by a conventional process (e.g., vacuum deposition) is employed. As the cathode, a highly conductive metal of low work function (e.g., magnesium, lithium, aluminum, silver) is used. In the case where magnesium is employed as the cathode, for improving the adhesion to a film for organic EL devices, magnesium may be deposited together with a small amount of silver (e.g., 1 to 10% by weight).

There is no particular restrictions on the process for the production of the organic electroluminescence device of the present invention, and conventional ones are adoptable. For example, the organic layer (e.g., light-emitting layer) is formed by forming a coat being the aforementioned transparent electrode (e.g., ITO electrode) on a transparent substrate and then applying or casting a coating fluid comprising the compound of the formula (I) in a conventional manner (e.g., spin coating, casting). The organic electroluminescence device is fabricated by further forming a cathode on the organic layer by vapor deposition or other means. If necessary, the anode or the light-emitting layer may be laminated with a layer or layers having an electron- and/or hole-transmitting function by such a conventional method as vapor deposition and coating.

Examples of the substrate are those transparent enough to transmit light emitted by the emission center compound (e.g., glass plates of soda glass, non-alkali glass, or quartz glass and sheets or films of polymers such as polyester, polysulfone, polyethersulfone). When fabricating a flexible organic EL device, the use of a polymer film is preferred.

Although the thickness of the organic EL device (e.g., the organic layer plus the electrodes) as a whole is not particularly limited, it is about 50 nm to 10 μm, preferably about 100 nm to 8 μm, and more preferably about 300 nm to 5 μm.

According to the present invention, since a nitrogen-containing heterocyclic compound having a specific structure is employed as the organic layer (particularly, light-emitting layer) of the organic EL device, it is possible to control the wavelength of light the organic EL device emits. Moreover, according to the present invention, in spite of the fact that the nitrogen-containing heterocyclic compound has a non-planar structure, the compound can emit light of relatively long wavelength (e.g., about 530 to 700 nm, yellow to red) and provides an organic EL device of high luminance and high durability.

The compound of the present invention can emit light by being irradiated with light or by the action of an electric field because it has a specific nitrogen-containing heterocycle. Therefore, the compound of the present invention is useful as a functional material typified by a fluorescent material and a material for display devices. Since a compound which emits light by the action of an electric field is particularly useful as an emission center compound for use in an organic electroluminescence device and is capable of emitting light within a wide wavelength range, it is possible to control the wavelength of light to be emitted by an organic EL device.

### EXAMPLES

The following example are intended to show the invention in further detail and should by no means be construed as defining the scope of the invention. Incidentally, analytical methods used in the examples of the present invention are shown below.

¹H-NMR spectra and ¹³C-NMR spectra were measured by using tetramethylsilane as an internal standard in deuterochloroform or deuterodimethylsulfoxide with use of a Varian Unity-plus 300 NMR spectrometer. Mass spectra were measured with use of a GCMS-QP5000 specrtometer (manufactured by Shimadzu Corporation). Melting points ware measured with use of a melting point apparatus (MP-21) manufactured by Yamamoto without correction.

UV/visible spectra and fluorescence spectra were determined by dissolving a sample in chloroform at the proportion of 1.5×10⁻⁵ mol/L and measuring the UV/visible and fluorescence spectra of the sample with use of a U-2010 spectrometer (manufactured by Hitachi, Ltd.) and a F-4500 fluorescence spectrometer (manufactured by Hitachi, Ltd.), respectively.

### Example 1

In the presence of a catalytic amount of acetic acid, to a system containing 0.07 mol of diaminomaleonitrile and 0.07 mol of acetylacetone was added 7 g of phosphorus pentoxide and allowed to react in 400 ml of a solvent (ethanol) at reflux temperature for 15 hours to give 2,3-dicyano-5,7-dimethyl-6H-1,4-diazepine. Ten mmol (10 mmol) of the diazepine compound thus obtained was reacted with 10 mmol of 4-methoxybenzaldehyde in a solvent (benzene) in the presence of piperidine to give 2,3-dicyano-5,7-bis(2-4-methoxyphenyl) ethenyl)-6H-1,4-diazepine as the object compound (compound 1).

Melting point: 259 to 260° C.

NMR (CDCl₃) 7.54 (2H, dd, J=15.9, 3, 2CH), 7.49 (4H, dd, J=8.7, ph), 6.76 (2H, dd, J=15.9, 3, 2CH), 6.93 (4H, dd, J=8.7, 2.4, ph), 3.85 (6H, s, 2CH₃)

NMR (d₆-DMSO) 8.05 (2H, d, J=16.5, 2CH), 7.75 (4H, d, J=7.2, ph), 7.00 (4H, d, J=7.2, ph), 7.00 (2H, d, J=16.5, 2CH), 5.35 (1H, broad, CH) 3.81 (6H, s, 2CH₃), 2.05 (1H, broad, CH)

Mass spectra (m/e): 408(M+)

Peak wavelength of the absorption spectra (uv): λmax 434 nm (extinction coefficient εmax 24200)

Peak wavelength of the fluorescence spectra: Fmax 542 nm

Elemental analysis (C₂₅H₂₀N₄O₂): Calculated value: C=73.51%; H=4.94%; N=13.72% Found value: C=74.00%; H=5.12%; N=13.72%

### Example 2

The object compound 2,3-dicyano-5,7-bis(2-(4-dimethylaminophenyl)ethenyl)-6H-1,4-diazepine (compound 2) was obtained in the same manner as in Example 1 with the exception that 4-dimethylaminobenzaldehyde was used in lieu of 4-methoxybenzaldehyde.

Melting point: 260 to 262° C.

Mass spectra (m/e): 434(M+)

Peak wavelength of the absorption spectra ( uv): λmax 523 nm(εmax 31000)

Peak wavelength of the fluorescence spectra: Fmax 643 nm

Elemental analysis (C₂₇H₂₆N₆): Calculated value: C=74.63; H=6.03; N=19.34% Found value: C=73.92; H=6.07; N=18.93%

### Example 3

The object compound 2,3-dicyano-5,7-bis(2-(4-diethylaminophenyl)ethenyl)-6H-1,4-diazepine (compound 3) was obtained in the same manner as in Example 1 with the exception that 4-diethylaminobenzaldehyde was used in lieu of 4-methoxybenzaldehyde.

Melting point: 209 to 211° C.

NMR (d₆-DMSO) 7.93 (2H, d, J=16, 2CH), 7.81 (4H, d, J=9, ph), 6.79 (2H, d, J=16, 2CH), 6.68 (4H, d, J=9, ph), 3.40 (8H, q, J=7, 4CH₂), 2.09 (1H, s, CH), 1.09 (12H, t, J=7, 4CH ₃)

Mass spectra (m/e) 490(M+);

Peak wavelength of the absorption spectra (uv): λmax 539 nm (εmax 33400)

Peak wavelength of the fluorescence spectra: Fmax 653 nm Elemental analysis (C₃₁H₃₄N₆): Calculated value: C=75.89; H=6.98; N=17.13% Found value: C=76.34; H=7.30; N=17.12%

### Example 4

The object compound 2,3-dicyano-5,7-bis(2-(N-ethyl-3-carbazolyl)ethenyl)-6H-1,4-diazepine (compound 4) represented by the following formula was obtained in the same manner as in Example 1 with the exception that N-ethyl-3-carbazolylaldehyde represented by the following formula was used in lieu of 4-methoxybenzaldehyde.

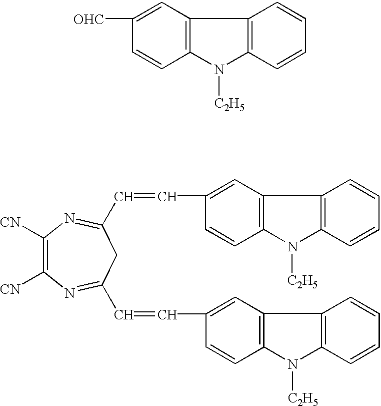

Melting point 254 to 256° C.

Mass spectra (m/e): 582(M+)

Peak wavelength of the absorption spectra (uv): λmax 475 nm (εmax 28000)

Peak wavelength of the fluorescence spectra: Fmax 605 nm

Elemental analysis (C₃₉H₃₀N₆): Calculated value: C, 80.39; H, 5.19; N, 14.42% Found value: C, 78.54; H, 5.28; N, 13.74%

### Example 5

The object compound 2,3-dicyano-5,7-bis(julolidine-9-yl)ethenyl)-6H-1,4-diazepine (compound 5) represented by the following formula was obtained in the same manner as in Example 1 with the exception that an aldehyde represented by the following formula was used in lieu of 4-methoxybenzaldehyde.

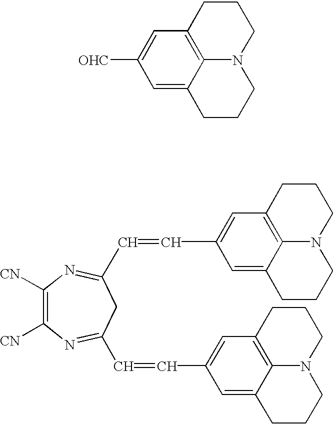

Melting point: >300° C.

Mass spectra (m/e): 538(M+)

Peak wavelength of the absorption spectra (uv): λmax 569 nm (εmax 30600)

Peak wavelength of the fluorescence spectra: Fmax 682 nm

Elemental analysis (C₃₅H₃₄N₆): Calculated value: C, 78.04; H, 6.36; N, 15.60% Found value: C, 77.36; H, 6.47; N, 14.99%

The measurement results of the absorption spectra and the fluorescence spectra of the compounds obtained from the above Examples are shown in Table 1.

TABLE 1

| | | | | | | Fmax − |
|---|---|---|---|---|---|---|
| | | | λmax | | Fmax | λmax |
| | n | Rings Z¹ and Z² | (nm) | εmax | (nm) | (nm) |
| Ex. 1 | 1 | methoxyphenyl | 433 | | 543 | 110 |
| Ex. 2 | 1 | dimethylaminophenyl | 523 | 31000 | 643 | 120 |
| Ex. 3 | 1 | diethylaminophenyl | 539 | 33400 | 653 | 114 |
| Ex. 4 | 1 | N-ethylcarbazolyl | | | | |
| Ex. 5 | 1 | 9-julolidinyl | 569 | 30600 | 682 | 113 |
| Ex. 6 | 2 | diethylaminophenyl | | | | |
| Comp. | 1 | −/diethylaminophenyl | 493 | 42400 | 591 | 98 |
| Ex. 1 | | | | | | |

### Examples 5 [Organic Electroluminescence Device]

A coating fluid was prepared by dissolving 150 mg of polymethylmethacrylate (PMMA: manufactured by Aldrich Chemical Company, Inc.), 100 mg of 2-(4-biphenyl)-5-(4-tert-butylphenyl)-1,3,4-oxadiazol (PBD: manufactured by Aldrich Chemical Company, Inc.), 2 mg of the compound (4) obtained in Example 2, and 50 mg of N,N′-diphenyl-N,N′-di(m-tolyl)-benzine (TDD: manufactured by Tokyo Kaseisha) in 20 ml of 1,2-dichloroethane. After forming a film of indium-tin-oxide (ITO) on a glass substrate, the coating fluid was applied on the ITO film by spin coating to form an organic layer of 100 nm thick. Thereafter, on the organic layer, an Al/Li electrode of 100 nm thick (manufactured by Kojundo Kagaku, K. K.; Li content 1% by weight) was made by vacuum deposition to give an organic electroluminescence device.

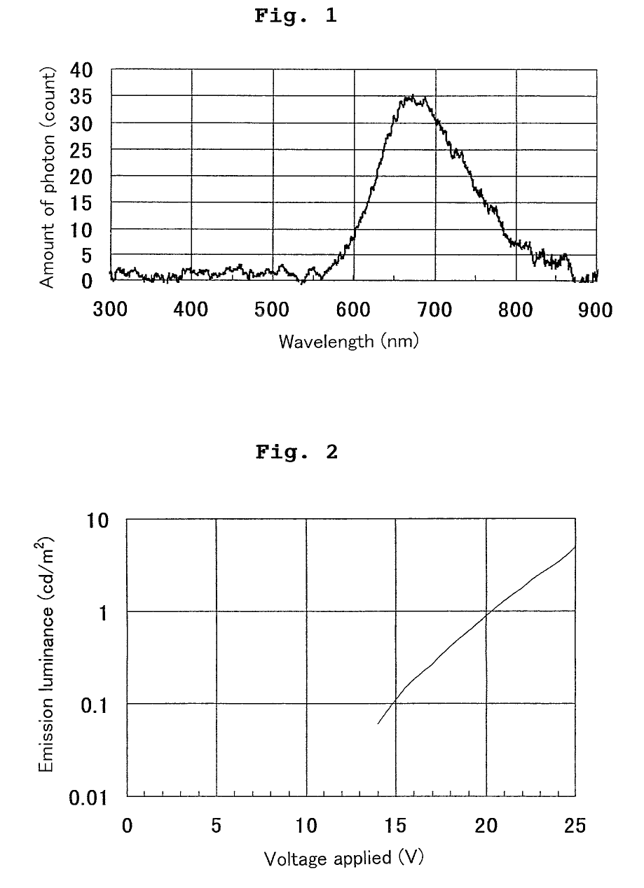

With the ITO electrode of the organic electroluminescence device obtained as anode and the Al/Li electrode as cathode, a direct current was applied between the both electrodes in the atmosphere thereby to make the device emit light (voltage applied: 20 V). As shown in FIG. 1, the peak wavelength of the emission spectrum was 673 nm (measured by a multichannel analyzer PMA-11 manufactured by Hamamatsu Photonics, K. K.,). The emission luminance was measured using a luminance detecting device (LS-110 manufactured by Minolta Co., Ltd.). FIG. 2 is a graph showing the value of luminance versus voltage applied.

What is claimed is:

1. A nitrogen-containing heterocyclic compound represented by the following formula (I): 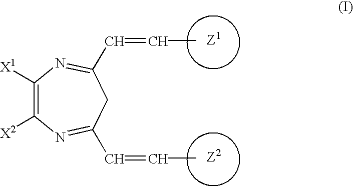
wherein X¹ and X² are the same or different, each representing an electron attractive group; and the rings Z¹ and Z² are the same or different, each representing a hydrocarbon ring which may have a substituent or a heterocycle which may have a substituent.

2. A nitrogen-containing heterocyclic compound according to claim 1, wherein the electron attractive group is selected from a cyano group, a carbonyl group, an acyl group, and a carboxyl group.

3. A nitrogen-containing heterocyclic compound according to claim 1, wherein at least one of the groups X¹ and X² is a cyano group.

4. A nitrogen-containing heterocyclic compound according to claim 1, wherein each of the rings Z¹ and Z² is bonded to the adjacent C═C bond to form a conjugated system.

5. A nitrogen-containing heterocyclic compound according to claim 1, wherein the rings Z¹ and Z² are the same or different, each representing an aromatic ring having an electron donative group, or a condensed ring which has an electron donative group and is condensed an aromatic hydrocarbon ring with a heterocycle having a nitrogen atom as a hetero atom.

6. A nitrogen-containing heterocyclic compound according to claim 1, wherein the groups X¹ and X² are cyano groups, and the rings Z¹ and Z² are the same or different, each representing a benzene ring having an electron donative group; or a condensed ring having an electron donative group, in which a benzene ring is condensed with a heterocycle having a nitrogen atom as a hetero atom, and wherein each of the electron donative groups is selected from a hydroxyl group, an alkoxy group, an amino group and an N-substituted amino group.

7. A nitrogen-containing heterocyclic compound according to claim 6, wherein the heterocycle constituting the condensed ring is 5- or 6-membered heterocycle.

8. A nitrogen-containing heterocyclic compound according to claim 1, wherein the substituent of rings Z¹ and Z² are at least one member selected from the group consisting of an alkyl group, a cycloalkyl group, an aryl group, an aralkyl group, a halogen atom, a hydroxyl group, an alkoxy group, a mercapto group, an alkylthio group, a hydroxyalkyl group, a carbonyl group, a carboxyl group, an alkoxycarbonyl group, an acyl group, an acyloxy group, a cyano group, an amino group, an N-substituted amino group, a nitro group, and a sulfonyl group.

9. A nitrogen-containing heterocyclic compound according to claim 1, which is capable of emitting light by applying a light or an electric field.

10. A nitrogen-containing heterocyclic compound according to claim 9, which is capable of emitting light by applying a light having a wavelength of 360 to 860 nm.

11. A nitrogen-containing heterocyclic compound according to claim 6, which is capable of emitting a fluorescent light by being irradiated with light.

12. A process for producing a nitrogen-containing heterocyclic compound represented by the following formula (I): 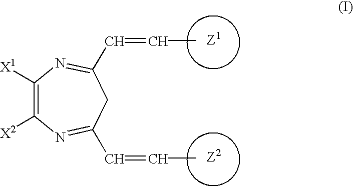
wherein X¹ and X² are the same or different, each representing an electron attractive group; and rings Z¹ and Z² are the same or different, each representing a hydrocarbon ring which may have a substituent, or a heterocylcle which may have a substituent
which comprises reacting a compound represented by the following formula (II): 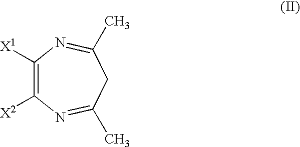
wherein the X¹ and X² have the same meanings as defined above
with a compound represented by the following formula (III): 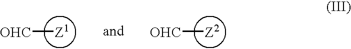
wherein the rings Z¹ and Z² have the same meaning as defined above.

13. An organic electroluminescence device, which comprises a pair of electrodes and an organic layer interposed therebetween, wherein the organic layer comprises a compound represented by the formula (I) recited in claim 1.

14. An organic electroluminescence device according to claim 13, wherein the organic layer has a light-emitting layer comprising a compound represented by the formula (I).

15. An organic electroluminescence device according to claim 14, wherein the organic layer has (1) a single layer structure composed of a light-emitting layer having at least one function selected from an electron-transporting function and a hole-transporting function, or (2) a layered structure composed of a layer having at least one function selected from an electron-transporting function and a hole-transporting function, and a light-emitting layer.

16. An organic electroluminescence device according to claim 13, wherein the organic layer comprises a compound represented by the formula (I) and an organic polymer having at least one function selected from an electron-transporting function and a hole-transporting function.
